(19) United States
(12) Patent Application Publication
(10) Pub. No.: US 2012/0109024 A1
(43) Pub. Date: May 3, 2012

(54) DEVICE AND ARRANGEMENT FOR DESTROYING TUMOR CELLS AND TUMOR TISSUE

(75) Inventor: Axel Erich Theuer, Kirchberg (DE)

(73) Assignee: ONCOWAVE MEDICAL GMBH, Wurzburg (DE)

(21) Appl. No.: 13/000,776

(22) PCT Filed: Jun. 25, 2009
(86) PCT No.: PCT/EP2009/004593

§ 371 Date: May 20, 2011

(30) Foreign Application Priority Data
Jun. 25, 2008 (DE) 10 2008 030 213.9

(51) Int. Cl. A61N 7/02

(52) U.S. Cl. 601/3

(57) ABSTRACT

Arrangements are provided for destroying tumor cells. According to one aspect, an arrangement includes a device having at least one high-frequency ultrasound generator for generating a thermally active, high-frequency oscillation, a plurality of first low-frequency ultrasound generators for generating a therapeutically active low-frequency oscillation, wherein the first low-frequency ultrasound generators each generate a different frequency, and a control system connected to the high-frequency ultrasound generator and to the first low-frequency ultrasound generators in such a way that the tumor cells are subjected to a high-frequency oscillation acting thermally on the tumor cells and to a low-frequency oscillation. The arrangement further includes a biopsy device having a plurality of individual receivers for tissue samples and a plurality of second low-frequency ultrasound generators operatively connected to the individual receivers for subjecting the tissue samples to an oscillation, wherein oscillations with the same frequency can be generated respectively by first and second low-frequency ultrasound generators.

### CROSS-REFERENCE TO RELATED APPLICATIONS

This application is a Section 371 of International Application No. PCT/EP2009/004593, filed Jun. 25, 2009, which was published in the German language on Dec. 30, 2009, under International Publication No. WO 2009/156156 A1 and the disclosure of which is incorporated herein by reference.

### BACKGROUND OF THE INVENTION

The invention relates to an arrangement and device for destroying tumor cells and tumor tissue.

German published patent application DE 100 23 457 A1 discloses an ultrasound instrument for therapeutic treatment of tumor diseases. It is known that tumor cells when stimulated by ultrasonic waves have an altered oscillation behavior compared to healthy cells. The rigidity and nature of the cytoskeleton, the viscosity of the cytoplasm, the plasma membrane and the nuclear fluid, the nucleus-to-plasma ratio, the osmotic pressure, the rigidity and nature of the extracellular matrix, the viscosity of the extracellular fluid, and the speed of the cellular aggregation process are oscillation-related cell parameters. Compared to healthy cells, malignant cells have an altered cytoskeleton, and invasive tumor cells therefore show a modification of their intermediary filaments. The nucleus-to-plasma ratio is shifted in favor of the cell nucleus. The nucleus of malignant cells is often changed in size and has an increased chromatin density.

Various therapy methods take advantage of this altered oscillation behavior. Thus, tumor cells can be stimulated with a resonant frequency inherent to them and can be destroyed by controlled treatment with ultrasound.

However, the resonant frequencies of the individual tumor cells are not uniform. Furthermore, the differences in the oscillation behavior of healthy cells and diseased cells are not consistently so great that it is possible to safely prevent damage of the healthy cells during conventional treatment with ultrasound or to at least reduce such damage. Although one can reduce the frequency range and the power of the ultrasound used, this reduction means that a large number of tumor cells are not covered by the treatment as the tumor cells are heterogeneous.

### BRIEF SUMMARY OF THE INVENTION

Proceeding from this prior art, the object of the present invention is to provide an arrangement and a device for improved treatment of tumor diseases.

In particular, the object is achieved according to a first aspect of the invention by a device for destroying tumor cells, wherein the device includes at least one high-frequency ultrasound generator for generating a thermally active, high-frequency oscillation, at least one low-frequency ultrasound generator for generating a therapeutically active low-frequency oscillation, and a control system connected to the high-frequency ultrasound generator and to the low-frequency ultrasound generator in such a way that the tumor cells are subjected to a high-frequency oscillation acting thermally on the tumor cells and to a low-frequency oscillation, wherein the low-frequency oscillation can be set by the control system to a frequency that destroys the tumor cell.

According to a second aspect of the invention, this object is achieved by providing an arrangement for destroying tumor cells that includes the above-described device according to the first aspect of the invention.

Alternatively, according to a third aspect of the invention, the arrangement includes a device having at least one high-frequency ultrasound generator for generating a thermally active, high-frequency oscillation, a plurality of first low-frequency ultrasound generators for generating a therapeutically active low-frequency oscillation, wherein the first low-frequency ultrasound generators each generate a different frequency or are adapted thereto, and having a control system connected to the high-frequency ultrasound generator and to the first low-frequency ultrasound generators in such a way that the tumor cells are subjected to a high-frequency oscillation acting thermally on the tumor cells and to a low-frequency oscillation.

In addition to the device according to the above first and second aspects, in each case for destroying tumor cells, the arrangement includes a biopsy device having a plurality of individual receivers for tissue samples and having a plurality of second low-frequency ultrasound generators operatively connected to the individual receivers for the purpose of subjecting the tissue samples to oscillations, wherein oscillations having the same frequency can be generated respectively by a first and second low-frequency ultrasound generator.

The arrangements according to the invention are based on the principle of correlating the oscillation frequencies, which the biopsy device can generate or use for testing tissue samples, with the oscillation frequencies the device uses to treat the tumor. As a result, the treatment successes are significantly improved, since a tumor-specific active frequency adapted to the relevant stage of the tumor can be determined in advance and can be used with the device.

The invention is therefore based on using two different ultrasound generators, that is a low-frequency ultrasound generator (LF ultrasound generator) and a high-frequency ultrasound generator (HF ultrasound generator). Destruction of the tumor cells, and consequently of the tumor tissue using this device, takes place by stimulating the tumor cells with a low-frequency oscillation generated by the LF ultrasound generator and matched to the appropriate tumor cells. Better treatment successes are achieved by heating the tumor cells to be treated in advance or simultaneously, in particular locally, by the HF ultrasound generator.

In particular, heating of the tumor cells or of the tumor tissue due to the high-frequency oscillation leads to better selective differentiation of the tumor cells from healthy cells. Local, targeted heating of the tumor cells sets up a temperature gradient between the healthy cells and the diseased cells. The temperature gradient ensures that the differences in the oscillation behavior of the diseased and healthy cells are reinforced. The target problem is solved more effectively due to the more clearly delimited oscillation behavior, as a sharper delimitation of the oscillation behavior of the diseased cells leads to an improvement in the selective stimulation of the diseased cells due to their exposure to low-frequency ultrasound. The advantage of the invention is that the high-frequency ultrasound generator creates the prerequisite for being able to introduce thermal energy limited locally to the diseased cells in the body and to do this in a simple and straightforward manner. As a result, the selection criterion, namely the oscillation behavior of the diseased and healthy cells, and thus the success of treatment, is significantly improved.

The device may include a holder in which the high-frequency ultrasound generator, in particular its ultrasound probe, and the low-frequency ultrasound generator, in particular its ultrasound probe, are held. Suitable positioning ability of the ultrasound generators and of their ultrasound probes, in particular of the HF ultrasound probe, is expedient for good success of the treatment. This positioning may take place in relation to the tissue to be treated by an appropriate holder. For this purpose, the ultrasound generators may be held in an opposing position in the holder.

The holder may be formed like a bell or a bowl and at least partially limit a treatment space. Thus, the holder may accommodate a medium, in particular a fluid that enables improved application of the high-frequency and low-frequency oscillations. In particular, reflections at the boundary surfaces (e.g., between air and human tissue), undesirable absorption (for example, prior to the entry of the sound waves into the tissue to be treated), and dispersion on entry of the sound into the tissue may be prevented or reduced. Due to the holder's design, it may function as a vacuum chamber or as an overpressure chamber and facilitate placement of the device on the tissue to be treated. The cavity behavior during ultrasound treatment may be influenced by a pressure control assigned to the chamber. In this case, cavitation phenomena are suppressed by adjusting an overpressure in the chamber and cavitation phenomena are supported by adjusting a vacuum.

The device may include a fluid pump connected to the treatment space via a fluid line for circulating and/or for introducing and/or for discharging a fluid. The vacuum for the vacuum chamber and the overpressure for the overpressure chamber may therefore be created by the fluid pump. Moreover, a medium or additive to the fluid may be introduced, as required, into the treatment space or may be discharged from it via the fluid line. These additives may, for example, include sonosensitizers.

The fluid pump may include a heating and/or cooling unit for adjusting a predetermined temperature in the treatment space. The success of the treatment may be further augmented by external heating up or cooling down of the tissue to be treated by the fluid. In particular, the selective stimulation which should be achieved on application of the low-frequency ultrasound may be improved.

At least the high-frequency ultrasound generator may comprise an acoustic lens which focuses the oscillations generated. Other focusing means are possible, for example an HF ultrasound probe with oscillation arrays, that is to say a plurality of controllable oscillation elements. The focusing HF ultrasound generator has the advantage that a spatially confined tumor tissue can be heated.

The control system may be adapted in such a manner that a high-frequency oscillation of at least 0.25 MHz (megahertz), in particular at least 0.5 MHz, in particular at least 0.8 MHz, in particular at least 1 MHz, can be generated with the high-frequency ultrasound generator. The HF ultrasound generator may be correspondingly optimized for the frequencies.

The control system may also be adapted in such a manner that the low-frequency ultrasound generator can be used to generate an oscillation that is variable within the range from one kHz (kilohertz) to 200 kHz, in particular 1 kHz to 150 kHz, in particular 5 kHz to 150 kHz, in particular 10 kHz to 150 kHz, in particular 16 kHz to 150 kHz, in particular 20 kHz to 150 kHz, in particular 20 kHz to 80 kHz, in particular 20 kHz to 40 kHz. The low-frequency oscillation should be adjustable in such a manner that it lies within range of the resonant frequency of the tumor cells to be treated. Thus a specific stimulation of these cells can be achieved.

The HF ultrasound generator may be connected to the holder in such a manner that the generator is height- and/or length-adjustable and/or is laterally adjustable and/or pivotable. The positioning may, therefore, be carried out in at least one axial direction, in particular in two or in three axial directions (space coordinates) and/or by pivoting. Thus, the HF-ultrasound generator may be positioned advantageously opposite the tumor cells to be treated. The adjustability of the HF-ultrasound generator's orientation opposite the holder is particularly advantageous if the holder is fixedly arranged on the body to be treated. Optimum positioning of the HF ultrasound generator opposite the tumor cells to be treated then takes place by coordinating the position and aligning the HF ultrasound generator in the holder.

The device for destroying tumor cells may include an ultrasound diagnostic unit, which comprises a diagnostic ultrasound generator and an evaluation unit for the localization of specific tumor cells. Thus, easy localization of the tumor cells may be ensured. Localization may be correlated with the position of the HF and/or LF ultrasound generator. The treatment success may be further augmented, in particular with exact positioning of the HF ultrasound generator.

The control system may be coupled to an evaluation unit in such a manner that the HF ultrasound generator is controllable, such that the focus of the high-frequency oscillations can be adjusted to the localized tumor cells.

According to another aspect of the invention, the object referred to above is furthermore achieved by a device for destroying tumor cells having a high-frequency ultrasound generator and a control system, wherein the control system is adapted in such a manner that a high-frequency oscillation generated by the high-frequency ultrasound generator is pulsed or pulsable, such that the high-frequency oscillation is superimposed with a low-frequency oscillation. Thus, in place of the HF and the LF ultrasound generators there may be a single HF ultrasound generator, which by an appropriate control and/or configuration generates oscillations that are correspondingly high-frequency or low-frequency.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a simplified, cross-sectional view of a treatment bell in line with an embodiment according to the invention;

FIG. 2 a schematic diagram of control unit for operating the treatment bell according to FIG. 1;

FIG. 3 a cross-sectional view through a treatment bell in line with an embodiment according to the invention, taken perpendicular to the longitudinal axis of the treatment bell; and

FIG. 4 a cross-sectional view through the biopsy device of the arrangement in line with an embodiment according to the invention.

The same reference numerals are used in the following description for identical parts and parts acting in an identical manner.

### DETAILED DESCRIPTION OF THE INVENTION

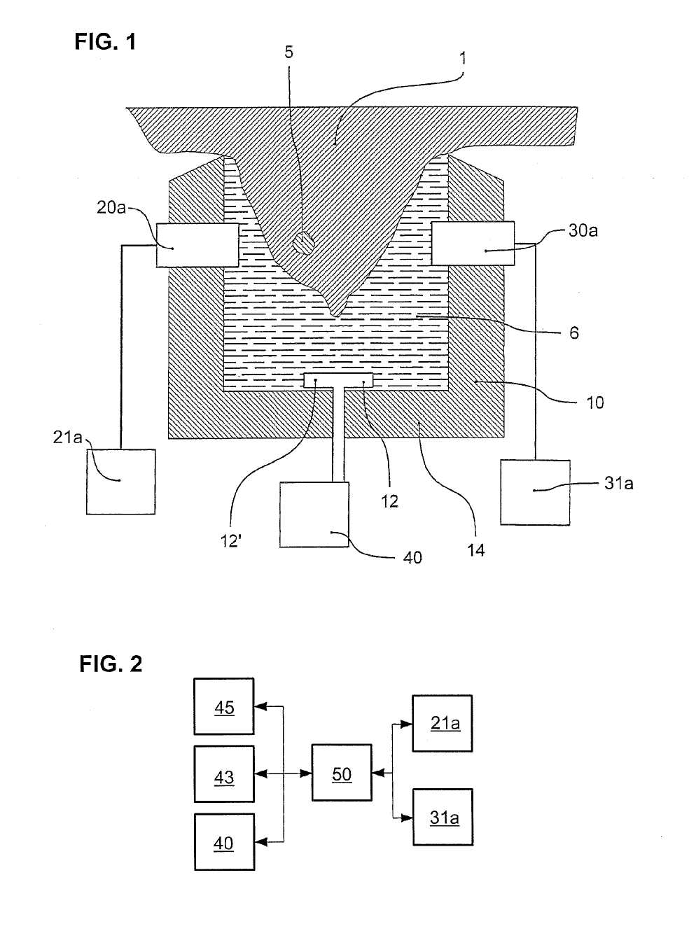

FIG. 1 shows a treatment bell 10 placed over tissue 1 to be treated, in the present case a female breast. Treatment bell 10 is configured to be cylindrical and is open towards one side. The opening is in air-tight contact with tissue 1. In the inner region of treatment bell 10 or in the treatment space there is a fluid 6 (e.g. water). For the application of high-frequency and low-frequency oscillations, treatment bell 10 includes an HF ultrasound probe 20a and an LF ultrasound probe 30a, which are disposed in the side walls of treatment bell 10.

HF ultrasound probe 20a and LF ultrasound probe 30a are focusing transducer probes, which emit focused sound waves into the interior of treatment bell 10. HF ultrasound probe 20a and LF ultrasound probe 30a are aligned in such a manner and are excited such that sound fields are produced, the maximum power of which is located in a focused target area inside the treatment space. The target area includes tumor 5.

It is sufficient if only narrow band HF ultrasound probe 20a is focusing. The ultrasound dealt with here is thus a high-intensity focused ultrasound (HIFU). LF ultrasound probe 30a need not be focusing, as the wide band sound signal which it emits is selected such that only tumor cells and no healthy cells are stimulated. Refer to the Background section above with regard to the different stimulation behavior of tumor cells and healthy cells.

To ensure optimum orientation of HF ultrasound probe 20a and LF ultrasound probe 30a, the appropriate probes are disposed on treatment bell 10 so as to be mechanically movable. It is possible to adjust the height of the probes in relation to base 14 of treatment bell 10. Moreover, the probes can be moved in such a manner that they protrude further into the interior or treatment area of treatment bell 10.

In a further embodiment, it is conceivable to support HF ultrasound probe 20a and LF ultrasound probe 30a freely in such a manner that they may be aligned in any position.

Treatment bell 10 includes a fluid inlet 12 and a fluid outlet 12′ on base 14. Fluid 6 may be introduced into the treatment area via fluid inlet 12, while fluid 6 may be discharged from the treatment area via fluid outlet 12′. Fluid inlet 12 and fluid outlet 12′ are connected to a vacuum pump 40 which, among other things, is configured to generate a vacuum in the treatment area, as a result of which treatment bell 10 attaches itself to tissue 1 to be treated. As a result of this it is possible to achieve efficient fixation of treatment bell 10 in relation to tissue 1 to be treated. A different type of fixation, for example mechanical fixation (contact pressure), is possible.

Vacuum pump 40 may also include a degassing (venting) device 43 and a heating/cooling unit 45. Fluid 6 from the treatment area is circulated through heating/cooling unit 45 and venting device 43 by vacuum pump 40. Degassing device 43 filters gases out of fluid 6. Cavitations in fluid 6 may be prevented by using a degassed fluid 6. Degassing device 43 may also be used when filling the treatment area with fluid 6 in order to provide degassed fluid 6 there from the outset. Pump 40 is also suitable as an overpressure pump. The cavitation behavior can be influenced by a control system of pump 40 and, in conjunction with this, by a pressure change in the treatment space.

Fluid 6 may be heated or cooled using heating/cooling unit 45. Tissue 1 is indirectly heated by heating or cooling fluid 6. An advantageous treatment may be achieved by changing the temperature of the tissue, in particular it is possible to improve selective treatment of the tumor cells by exposing them to low-frequency ultrasound, since at certain temperatures the characteristic oscillation behavior of healthy cells changes in favor of the selective stimulation.

The device also includes a control system 50 (FIG. 2) which is connected to vacuum/overpressure pump 40, degassing device 43, heating/cooling unit 45, an HF generator 21a and an LF generator 31a. Control unit 50 may perform various open and closed loop control tasks when treating tumor 5. In particular, it adjusts HF generator 21a and LF generator 31a in such a manner that the ultrasound probes connected to them, that is HF ultrasound probe 20a and LF ultrasound probe 30a, generate an appropriate low-frequency or high-frequency oscillation. Furthermore, control unit 50 may control the temperature of fluid 6 and the vacuum or overpressure in the treatment area.

The terms “high-frequency ultrasound generator” and “low-frequency ultrasound generator” are to be understood in the sense of an ultrasound unit that includes an ultrasound probe 20a, 30a and an ultrasound generator 21a, 31a, wherein ultrasound probe 20a, 30a is connected to the treatment unit or holder 10. Since ultrasound probes 20a, 30a are excited by ultrasound generators 21a, 31a and emit ultrasonic oscillations, ultrasound probes 21a, 31a may also be termed as oscillation probes or ultimately also as a kind of generator.

In a further embodiment (not illustrated), treatment bell 10 includes a diagnostic device, by which the position of tumor 5 can be determined precisely. This diagnostic device may deliver appropriate signals to control unit 50. Detection of the position of tumor 5 by the diagnostic device may also be carried out by way of appropriate ultrasound signals. The person skilled in the art should be aware of relevant detection methods.

In a further embodiment, control unit 50 may activate HF ultrasound probe 20a and/or LF ultrasound probe 30a in such a manner that it or they may be used as a diagnostic unit. It is conceivable to align HF ultrasound probe 20a and the LF ultrasound probe 30a automatically according to the position determined by the detection device.

It is important for the success of the treatment of tumor 5 to adjust a low-frequency oscillation that is suitable for triggering resonance phenomena, which then initiate cell death in the largest possible number of tumor cells. The excitation oscillation appropriate for this varies approximately within a range between 10 and 40 kHz. Since the appropriate excitation oscillation is tumor-specific, it is possible by a preliminary determination of the optimum frequency or of the optimum frequency range to achieve significantly better treatment success. To do this, tumor tissue is removed and analyzed in a frequency determination unit. This frequency determination unit includes a monocrystalline oscillation probe (frequency range 16 to 80 kHz) which is excited by way of an appropriate generator. This monocrystalline oscillation probe is configured two-dimensionally, in such a manner that there is space for a plurality of recesses on its upper side. The tissue sample may be distributed in these recesses. An oscilloscope connected to the monocrystalline oscillation probe determines the oscillation frequency of the monocrystalline oscillation probe when excited by the generator. A control system may vary this oscillation frequency until cell death of the tumor cells occurs. The frequency thus determined may be used for treatment in treatment bell 10. There are numerous conceivable methods which may be used to determine cell death of the tumor cells and the appropriate stimulation frequency. It is advantageous to operate the frequency determination unit in such a manner that cell death can be ascertained visually. For example, bursting of the tumor cells may be observed.

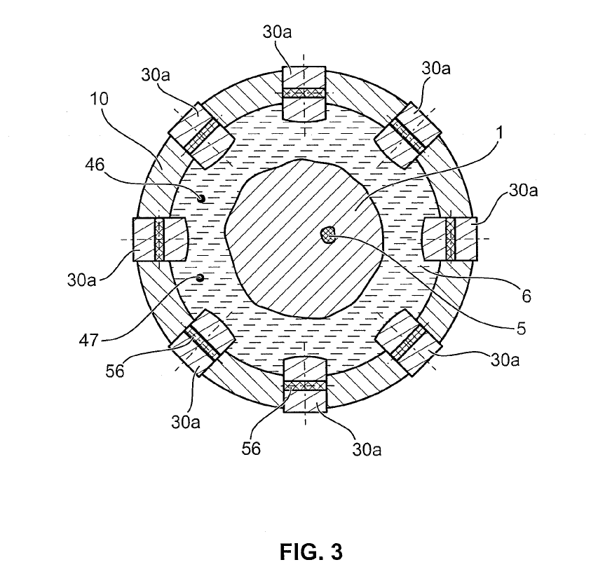

FIG. 3 illustrates a cross-section through a treatment bell 10 or generally through a treatment device for destroying tumor cells, wherein the cross-section runs perpendicular to the centerline of treatment bell 10 (unlike the cross-section according to FIG. 1 which runs along the centerline). High-frequency ultrasound probe 20a and associated high-frequency generator 21a are disposed outside the cross-sectional plane and are therefore not illustrated in FIG. 3. The treatment bell according to FIG. 3 includes a plurality of low-frequency ultrasound probes 30a disposed on the bell's circumference, in particular first low-frequency ultrasound probes 30a. The arrangement of first low-frequency ultrasound probes 30a should be considered as being by way of example. Other positions of individual probes 30a are possible. Eight probes 30a are provided in the embodiment according to FIG. 3. A different number of probes, for example within a range from two to sixteen, is possible. More than sixteen probes are also possible. Each probe 30a has an oscillation element 56, for example a piezoelectric element, that is or can be activated by a control system (not illustrated) or control unit 50. Individual first low-frequency ultrasound probes 30a each generate a different frequency. It is also possible for each of individual first low-frequency ultrasound probes/generators 30a, 31a to operate in a different frequency band. Tumor 5 can be exposed to the specific frequency intended for treatment by activating a specific low-frequency ultrasound probe 30a.

Disposed in treatment bell 10 is a treatment medium 6 which improves transmission of the oscillation from individual probes 30a to tumor 5. Fluid 6 may be tempered, in particular cooled, in order to obtain the greatest possible difference in temperature between healthy breast tissue 1 and the tumor heated locally by high-frequency ultrasound probe/generator 20a, 21a (not illustrated). For this, treatment bell 10 has a tempering unit (not illustrated), in particular a cooling unit.

In addition, temperature sensors 46 and pressure sensors 47 are provided to measure the temperature and pressure of fluid 6. For the rest, the features described in connection with the embodiment according to FIGS. 1 and 2 are also provided for the treatment bell according to FIG. 3.

Individual first low-frequency ultrasound probes 30a have the advantage that it is possible to select the appropriate oscillation frequency for the treatment of tumor 5 very quickly without elaborate adjustment. It is also possible to replace individual low-frequency ultrasound probes 30a by one or a plurality of low-frequency ultrasound probes operating on a wide bandwidth, which also enable the selection or activation of a tumor-specific oscillation frequency.

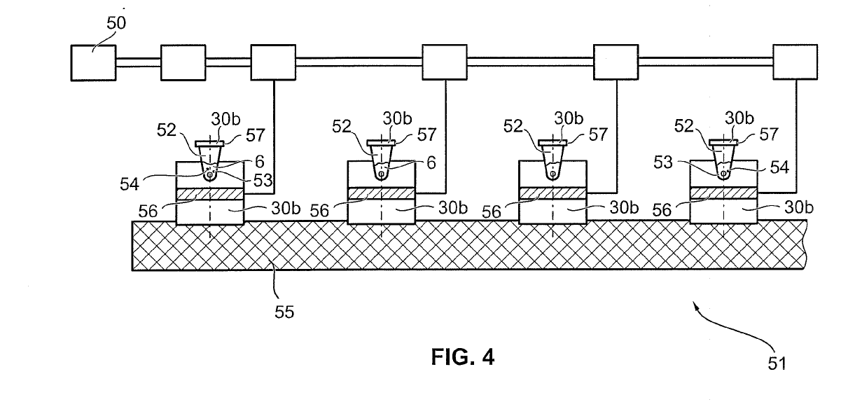

The device according to FIG. 3 is disclosed and claimed both independently of and also together with an arrangement for destroying tumor cells, which further includes a biopsy device illustrated in FIG. 4. The biopsy device according to FIG. 4 is configured in a similar manner to treatment bell 10 according to FIG. 3, insofar as the biopsy device has a plurality of second low-frequency ultrasonic oscillation probes 30b, wherein oscillations having the same frequency can be generated in each case by a first and second low-frequency ultrasound generator or oscillation probe 30a, 30b. This has the advantage that tissue samples of the tumor to be treated can be tested, prior to carrying out the treatment, with respect to the most effective oscillation frequency possible. The effective oscillation frequency thus found is then applied by corresponding first low-frequency ultrasound generators 30a of treatment bell 10.

Biopsy device 51 includes an insulation board 55 on which is arranged a plurality of second low-frequency ultrasound probes 30b, in particular in the same number as first low-frequency ultrasound probes 30a. FIG. 4 shows four of the total eight oscillation probes 30b, whereby the invention is not restricted to the number of eight probes. Each oscillation probe 30b includes an oscillation element 56, in particular a piezoelectric element activated by control system 50. In addition, an individual receiver 52 in which is disposed a physiological fluid 6 or a fluid 6 is connected to each oscillation probe 30b. A tissue sample of tumor 5 to be treated resides in fluid 6 during use. Individual receiver 52 is sealed by a cover 57. In addition, sensors, in particular an acceleration sensor 53 and a temperature sensor 54 are attached to individual receiver 52.

Control system 50 may be connected to both low-frequency ultrasound probes 30b of biopsy device 51 and also to low-frequency ultrasound probes 30a. This has the advantage that after determination of an optimum or effective frequency, the control unit can be switched over by relevant second low-frequency ultrasonic oscillation probe 30b of the biopsy device to relevant first low-frequency ultrasound probe 30a of the device, which has the same frequency as test probe 30b. This has the advantage that after determining the effective frequency it can be used for treatment without any time delay. A test result that is as unbiased as possible is used as the basis for the treatment of tumor 5 since the oscillation behavior of the tumor cells changes quickly in terms of time, in particular shortly before metastasis due to the formation of stiffening protein filaments.

One or a plurality of wide-band low-frequency ultrasound generators may be used in place of individual low-frequency ultrasonic oscillation probes 30b, which each have a specific test frequency or test frequency band. The effective oscillation frequency determined by the wide-band test probes is then used as the basis for the treatment probes of treatment bell 10.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1.-14. (canceled)

15. An arrangement for destroying tumor cells comprising:
a biopsy device having a plurality of individual receivers for tissue samples and a plurality of second low-frequency ultrasound generators operatively connected to the individual receivers for subjecting the tissue samples to an oscillation, wherein oscillations with the same frequency are generated respectively by a first and a second low-frequency ultrasound generator; and further comprising one of the following devices:
a first tumor destroying device having at least one high-frequency ultrasound generator for generating a thermally active, high-frequency oscillation, at least one low-frequency ultrasound generator for generating a therapeutically active low-frequency oscillation, and a control system connected to the high-frequency ultrasound generator and to the low-frequency ultrasound generator, such that the tumor cells are subjected to a high-frequency oscillation acting thermally on the tumor cells and to a low-frequency oscillation, wherein the low-frequency oscillation is set by the control system to a frequency that destroys the tumor cells; or
a second tumor destroying device having at least one high-frequency ultrasound generator for generating a thermally active, high-frequency oscillation, a plurality of first low-frequency ultrasound generators for generating a therapeutically active low-frequency oscillation, wherein the first low-frequency ultrasound generators each generate a different frequency or are adapted thereto, and having a control system connected to the high-frequency ultrasound generator and to the first low-frequency ultrasound generators, such that the tumor cells are subjected to a high-frequency oscillation acting thermally on the tumor cells and to a low-frequency oscillation.

16. The arrangement according to claim 15, wherein the control system is connected to the second low-frequency ultrasound generators.

17. A device for destroying tumor cells comprising at least one high-frequency ultrasound generator for generating a thermally active, high-frequency oscillation, at least one low-frequency ultrasound generator for generating a therapeutically active low-frequency oscillation, and a control system connected to the high-frequency ultrasound generator and to the low-frequency ultrasound generator, such that the tumor cells are subjected to a high-frequency oscillation acting thermally on the tumor cells and to a low-frequency oscillation, wherein the low-frequency oscillation is set by the control system to a frequency that destroys the tumor cells.

18. The device according to claim 17, further comprising a holder in which the high-frequency ultrasound generator and the low-frequency ultrasound generator are held.

19. The device according to claim 18, wherein the holder has a bell or bowl shape and at least partially limits a treatment space.

20. The device according to claim 19, further comprising a fluid pump connected to the treatment space via a fluid line for circulating and/or for introducing and/or for discharging a fluid.

21. The device according to claim 20, wherein the fluid pump includes a heating and/or cooling unit for adjusting a predetermined temperature in the treatment space.

22. The device according to claim 17, wherein at least the high-frequency ultrasound generator comprises an acoustic lens which focuses the oscillation generated.

23. The device according to claim 17, wherein the control system is adapted to cause the high-frequency ultrasound generator to generate a high-frequency oscillation of at least 0.25 MHz, optionally at least 0.5 MHz, optionally at least 0.8 MHz, optionally at least 1 MHz.

24. The device according to claim 17, wherein the control system is adapted to cause the low-frequency ultrasound generator to generate an oscillation variable within a range from 1 kHz to 200 kHz, optionally 1 kHz to 150 kHz, optionally 5 kHz to 150 kHz, optionally 10 kHz to 150 kHz, optionally 16 kHz to 150 kHz, optionally 20 kHz to 120 kHz, optionally 20 kHz to 80 kHz, optionally 20 kHz to 40 kHz.

25. The device according to claim 18, wherein the high-frequency ultrasound generator is connected to the holder such that the high-frequency ultrasound generator is height- and/or length-adjustable and/or is laterally adjustable and/or pivotable.

26. The device according to claim 17, further comprising an ultrasound diagnostic unit comprising a diagnostic ultrasound generator and an evaluation unit for localization of specific tumor cells.

27. The device according to claim 26, wherein the control system is coupled to the evaluation unit such that the high-frequency ultrasound generator is controllable to adjust a focus of the high-frequency oscillation to localized tumor cells.

28. A device for destroying tumor cells comprising a high-frequency ultrasound generator and a control system, wherein the control system is adapted such that a high-frequency oscillation generated by the high-frequency ultrasound generator is pulsed or pulsable to superimpose the high-frequency oscillation with a low-frequency oscillation.
